(19) United States
(12) Patent Application Publication
(10) Pub. No.: US 2021/0406801 A1
(43) Pub. Date: Dec. 30, 2021

(54) A METHOD AND SYSTEM FOR COMPETENCY BASED ASSESSMENT

(71) Applicant: Robert Peter Smart, Ottawa (CA)

(72) Inventor: Robert Peter Smart, Ottawa (CA)

(21) Appl. No.: 16/625,337

(22) PCT Filed: Jun. 22, 2018
(86) PCT No.: PCT/CA2018/050763

§ 371 Date: Dec. 20, 2019

(51) Int. Cl. G06Q 10/06; G06Q 10/10

(52) U.S. Cl. CPC G06Q 10/06393; G06Q 10/105

(57) ABSTRACT

The present invention is directed to a system and method for competency assessment and verification. The system includes a task and criteria module for defining tasks and criteria, an evidence module for receiving task and criteria evidence and an assessment module for assessing tasks and criteria based on the received evidence, in a web-based environment. The evidence module may include a documentary component and a video component for receiving documentary and video evidence. The assessment module may include an evidence indexing or annotating component for location indexing of documentary evidence and time indexing of video evidence.

### FIELD OF THE INVENTION

The present invention pertains to the field of assessment and verification of competencies, and in particular to a method and system for electronic assessment and verification of competencies contained within an electronic portfolio.

### BACKGROUND

Historically, an evaluation of a candidate for employment, accreditation or certification would be based on the candidates' education and qualifications, e.g. does the candidate know what to do. Recently, there has been an increase in evaluating candidates based on their competency, e.g. does the candidate know how to do it.

Competence has been defined in international Organization for Standardization (ISO) Standard 19011.2011 as the ability to apply knowledge and skills to achieve intended results. When a candidate is evaluated based on that person's competency, his knowledge, skills, personal attributes and qualifications specific to the job duty and/or scope of certification is examined. On the other hand, when a candidate is evaluated based on that person's qualifications, the candidate's education and qualifications are examined rather than based on measurable competence. The following example further highlights the difference between competency and qualification. In response to the question. “Do you know how to drive a car?” A candidate response, “I have acquired a driving license but I am still not confident to drive a car.” This response demonstrates the candidate has the qualification, but not the competency, to drive a car.

Recognizing that competency-based evaluations, accreditations and certifications provide a more thorough evaluation than one based on qualifications alone, companies and organizations have started to require competency-based training and evaluations. For example. ISO/IEC Standard 17024 Conformity Assessment—General requirements for bodies operating certification of persons, mandates that the certification of persons should be based on the demonstration of competencies and not the demonstration of qualifications. ISO/IEC 17024 sets the requirements and the framework, at a global level, for the operation of Personnel Certification Bodies (PCB). By using ISO/IEC 17024, business, industry and other key stakeholders have recognized that competency-based certification is the optimum way of achieving confidence in persons certified by PCBs. In another example, in the Caribbean, five competency-based levels of certification have been articulated to the tradesman, craftsman, technician, technologist and engineer.

The assessment of competency including the assessment of the ability to preform a specific activity or function is also relevant in education, and in health and wellness Assessment of changes in competency can be used to monitor the effectiveness of training and/or treatment regimes and/or to monitor age-related decline or disease progression.

When examining or assessing the competency of a candidate, the assessment must be valid, reliable and independent. There is a need for an efficient competency-based assessment system that is valid, reliable and independent.

This background information is provided to reveal information believed by the applicant to be of possible relevance to the present invention. No admission is necessarily intended, nor should he construed, that any of the preceding information constitutes prior art against the present invention.

### SUMMARY

An object of the present invention is to provide a method and system for competency-based assessment. In accordance with an aspect of the present invention, there is provided a computer-implemented method of conducting a competency-based assessment of a candidate, the method comprising a) providing a database comprising a plurality of electronic profiles, wherein each electronic profile is for a specific candidate and comprises a candidate identifier and a competency level; b) updating an electronic profile of a specific candidate to include electronic evidence of achievement, wherein the evidence of achievement supports a specific competency; c) indexing or annotating the electronic evidence based on a criterion to obtain processed electronic evidence; and d) defining a level of competency for the specific competency based on the processed evidence of achievement.

In accordance with another aspect of the present invention, there is provided a computer-readable non-transitory storage medium storing instructions executable by a processor, the instructions comprising a set of instructions to access a database of a plurality of electronic profiles, wherein each electronic profile is for a specific candidate and composes a candidate identifier and a competency level and to update an electronic profile of a specific candidate to include electronic evidence of achievement, wherein the evidence of achievement supports a specific competency; a set of instructions for indexing or annotating the electronic evidence based on a criterion to obtain processed electronic evidence, and a set of instructions for defining a level of competency for the specific competency based on the processed evidence of achievement.

In accordance with another aspect of the present invention, there is provided an online system of conducting a competency-based assessment of a candidate, the system being configured to access a database of a plurality of electronic profiles, wherein each electronic profile is for a specific candidate and comprises a candidate identifier and a competency level and to update an electronic profile of a specific candidate to include electronic evidence of achievement, wherein the evidence of achievement supports a specific competency; to index or annotate the electronic evidence based on a criterion to obtain processed electronic evidence; and to define a level of competency for the specific competency-based on the processed evidence of achievement.

In accordance with another aspect of the present invention, there is provided a competency-based assessment system comprising an electronic profile module for identifying a candidate and competencies of interest, and submission of evidence of competency; a task and criteria module for defining tasks and criteria relating to various competencies; and an assessment module for assessing tasks and criteria based on the submitted evidence.

The method and system may be configured to output a level of a specific competency in the form of a grade or a pass/fail indication. In some embodiments, the method and system are further configured to provide guidance to improve the level of competency and/or feedback.

The candidate underassessment includes an individual including a human or non-human animal, an institute, an organization, a group of individuals including a team or a company. Individual candidates include patients including those undergoing treatment or not undergoing treatment, test subjects, trainees, students, employees, apprentices, candidate for employment or certification, athletes amongst others. The method and system provide for the entry of biographical information, the nature of which will depend of the type of assessment and the candidate, in some embodiments, the biographical information comprises one or more of name, address, title, age and image of the candidate. In some embodiments, the candidate under assessment is a computer entity or robotic candidate.

The system and method can be configured to assess an anonymous candidate or a known candidate. Accordingly, in some embodiments how a user and/or the system identify the candidate is dependent on if the candidate is to remain anonymous or not. A candidate identifier may be the candidate's name or official identifier, it may be user or candidate defined, it may be randomly generated or assigned sequentially

In some embodiments, the present invention is directed to a system for on-line competency assessment and verification The system includes an electronic profile module for identifying a candidate and the competencies of interest, and the submission of evidence of competency; task and criteria module for defining tasks and criteria relating to various competencies; and an assessment module for assessing tasks and criteria based on the received evidence, optionally in a web-based environment.

The system and method may be configured to assess a single competency per candidate or alternatively configured to assess more than one competency per candidate.

The specific competency assessed by the method and system include those that are user defined, are selected from a pre-set list, are requirements of an accreditation or program amongst others.

The one or more criteria used to assess a competency, in some embodiments, are predetermined based on competency assessed, are optionally selected from a list or set of criteria or may be user or evaluator defined.

The received evidence may include a documentary or video evidence. Other evidence that may be inputted into the system and method includes text, electronic documents, images, videos, blog entries, multimedia presentation, audio recordings, health and fitness data and map coordinates. In some embodiments, the electronic evidence of an achievement is a result from a standardized test.

The assessment module includes an evidence indexing or annotating component for location indexing of evidence including time indexing of video or audio evidence.

In some embodiments, the assessment module or the assessment step of the method provides for the indexing or annotating of the electronic evidence and facilitate the reviewing of the electronic evidence and bookmarking, highlighting a pad of the evidence, time indexing and/or meta data tagging of the evidence. Indexing and annotating the evidence may be completed using a mouse, touch pad or touch screen amongst other means known in the art. Optionally details and/or links to bookmarks, highlights, time index, and/or meta data tagging are aggregated into a list in a dedicated section of a user interface in some embodiments, the dedicated section is searchable and may be in the form of a bookmark pane, pop-up box, or separate dialog box. Appropriate user interfaces are known in the art.

In some embodiments, each bookmark in the bookmark pane is assigned one or more actions. The actions include but are not limited to going to a specific page or passage in the document, going to a specific frame in the video or point in the audio or opening a separate file including an image file or text file, optionally in a new pane.

In some embodiments, the assessment method and system can be used to assess for a change in the level of competency over time. The change in competency includes no change, an improvement in a competency or a decline or decrease in a competency. Accordingly, the assessment method and system may be used to assess responses or the effectiveness of training, the effectiveness of a treatment regime, age related changes and/or disease progression. In some embodiments, the assessment method and system is configured to assess cognitive decline or impairment.

It is to be understood that both the foregoing general description and the following detailed description are exemplary and explanatory only and are not restrictive of the invention as claimed. The accompanying drawings, which are incorporated herein by reference, and which constitute a part of this specification, illustrate certain embodiments of the invention and, together with the detailed description, serve to explain the principles of the present invention.

### BRIEF DESCRIPTION OF THE FIGURES

Further features and advantages of the present invention will become apparent from the following detailed description, taken in combination with the appended drawings, in which:

FIG. 1 is a flow chart depicting the competency assessment system according to an embodiment of the present invention;

FIG. 2 is a diagram of a screen print depicting a summary of the status and detail of a user according to an embodiment of the present invention;

FIG. 3 is a diagram of a screen print depicting a graphic summary of the accomplishments of a user according to an embodiment of the present invention;

FIG. 4 is a diagram of a screen print depicting a video evidence submission having a criteria time tracking notation therein according to an embodiment of the present invention;

FIG. 5 is a diagram of a screen print depicting a user submission in the form of a certification according to an embodiment of the present invention;

FIG. 6 is a diagram of a screen print depicting a detailed view of a user's tasks and criteria portfolio according to an embodiment of the present invention;

FIG. 7 is a diagram of a screen print depicting an example of how guidance can be provided to a user according to an embodiment of the present invention;

FIG. 8 is a diagram of a screen print depicting user submissions and video evidence as evidence according to an embodiment of the present invention;

FIG. 9 is a diagram of a screen print depicting a listing of tasks and criteria along with video and written documentation as evidence according to an embodiment of the present invention;

FIG. 10 is a diagram of a screen print depicting a video submission having an assessment of sufficient according to an embodiment of the present invention;

FIGS. 11a and 11b are diagrams of a screen print depicting a video submission having an assessment of insufficient according to an embodiment of the present invention;

FIG. 12 is a diagram of a screen print depicting a Signature signoff according to an embodiment of the present invention;

FIG. 13 is a diagram of a screen print depicting a time indexing feature of the present invention according to an embodiment of the present invention;

FIG. 14 is a diagram of a screen print depicting a written document as evidence and showing a highlighted portion thereof used for tracking of deletion of tasks in criteria according to an embodiment of the present invention; and

FIGS. 15a and 15b are diagrams of a screen print depicting a dashboard of a user according to an embodiment of the present invention.

### DETAILED DESCRIPTION

The present invention pertains to the field of assessment and verification of competencies, and in particular to a system for electronic assessment and verification of competencies contained within an electronic portfolio.

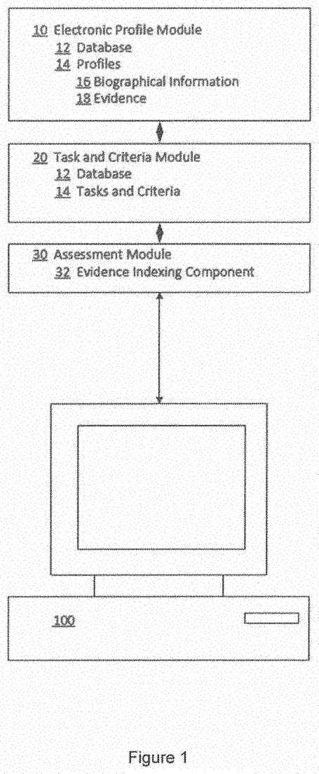

An embodiment of the present invention is shown in FIG. 1 as system 1. System 1 composes an electronic profile module 10, a task and criteria module 20. and an assessment module 30, in an on-line, web-based environment 100. An on-line web-based environment 100 is defined as any suitable database, hardware, software, network or application, including but not limited to, intranets, internets, or any other web-based environment available on a single computer, a network of computers, a local server, one or more hand-held mobile devices, or on an intranet or on the internet and world-wide-web.

In general operation, this embodiment of the system allows a user to create an electronic profile 14 of a candidate, which could be themselves or another person. The profile identifies the person's competency level of various abilities. Competencies can be defined as a set of context-specific qualities, such as but not limited to, related knowledge, skills, abilities, and attributes that form a candidate's ability to perform a job. Competencies can be correlated with superior job performance and can be used as a standard against which to measure job performance as well as to develop, recruit, and hire employees.

The competencies in this embodiment include various tasks and criteria 24. The tasks and criteria 24 are stored in the task and criteria module 20. Evidence 18, which is submitted by or on behalf of the person, is evaluated through the assessment module 30. This embodiment of the system 1 may be capable of proving desirable standards that a candidate has established a competency in an ability in an open and transparent manner. Overall, this embodiment of system 1 allows for the assessment of person's competency in an ability, not whether someone has a specific credential i.e. whether someone has the ability, e.g. know how, to perform a job duty as opposed to just having a degree or diploma or certificate.

Electronic profile module 10 may comprise a database 12 of electronic profiles 14 of one or more candidates. An electronic profile 14 can be defined as a record that provides actual evidence of achievement and may contain biographical information 16 of a candidate in the case of where the candidate is a person, such as but not limited to name, address, title, age, and Image of person; and may also contain a collection of electronic evidence 18 of achievement, such as but not limited to input text, electronic documents, images, videos, blog entries, multimedia presentations, and hyperlinks. Where the candidate is other than an individual person appropriate biographical information may be provided as would be apparent to one skilled in the art.

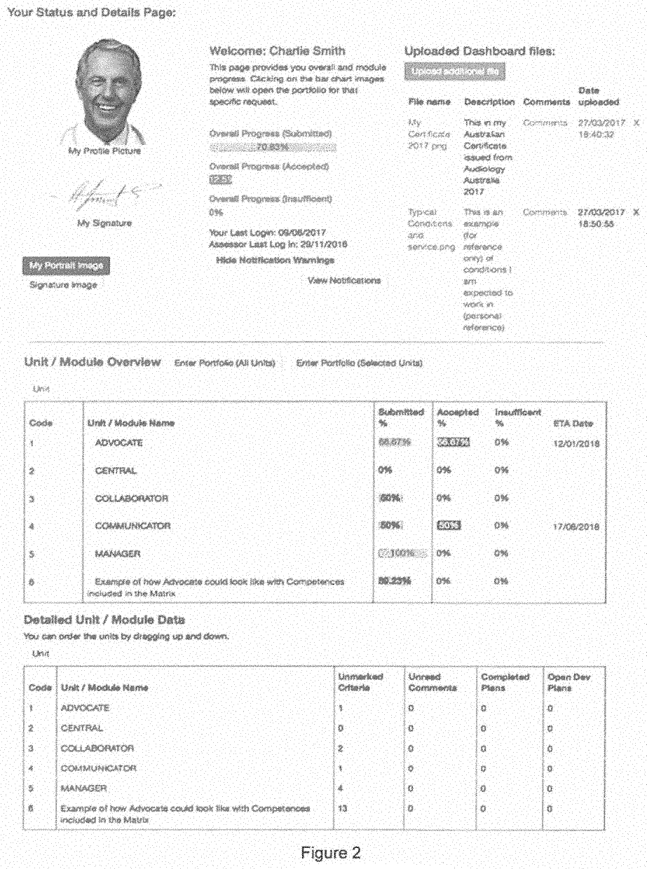

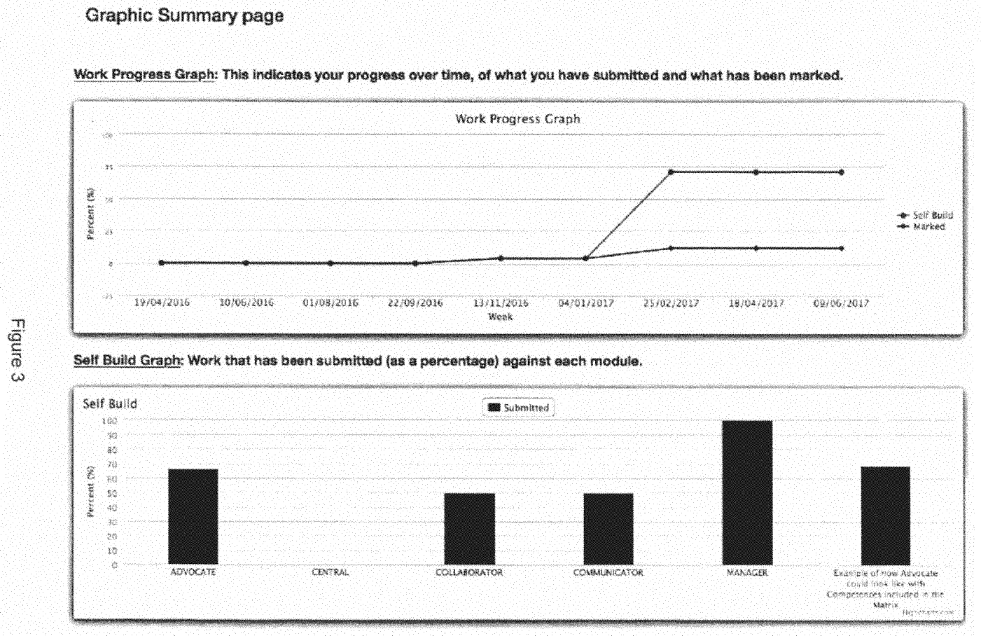

An embodiment of an electronic profile 14 is shown in screen shot FIG. 2 which depicts the name of a candidate associated with the profile, the evidence that has been submitted by the person, a listing of the various competencies the person is attempting to satisfy and the current progress toward satisfying those competencies.

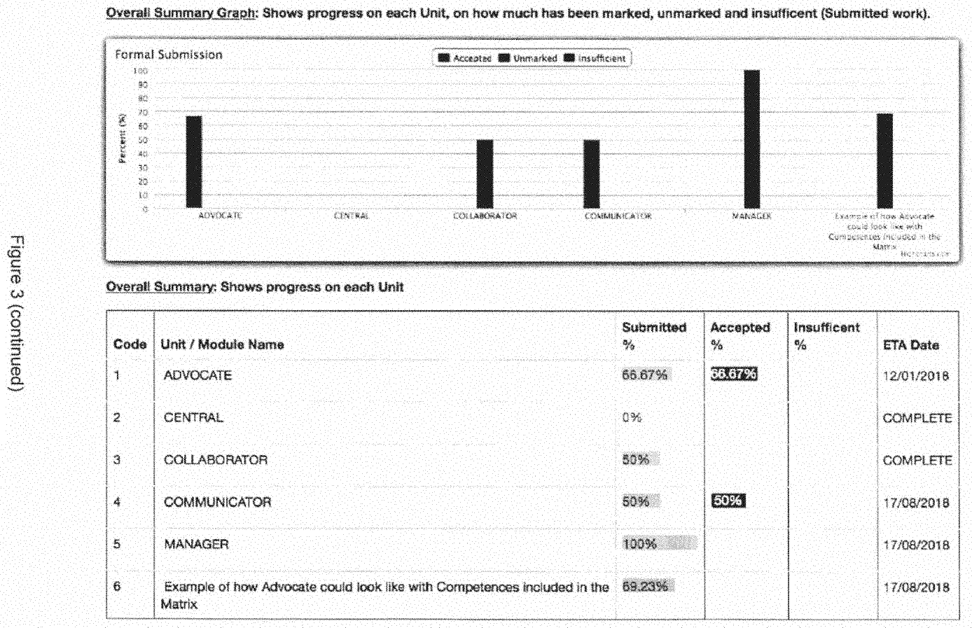

Another embodiment of an electronic profile 14 is shown in screen shot FIG. 3 which depicts graphical representation of the current progress toward satisfying a candidate's competencies.

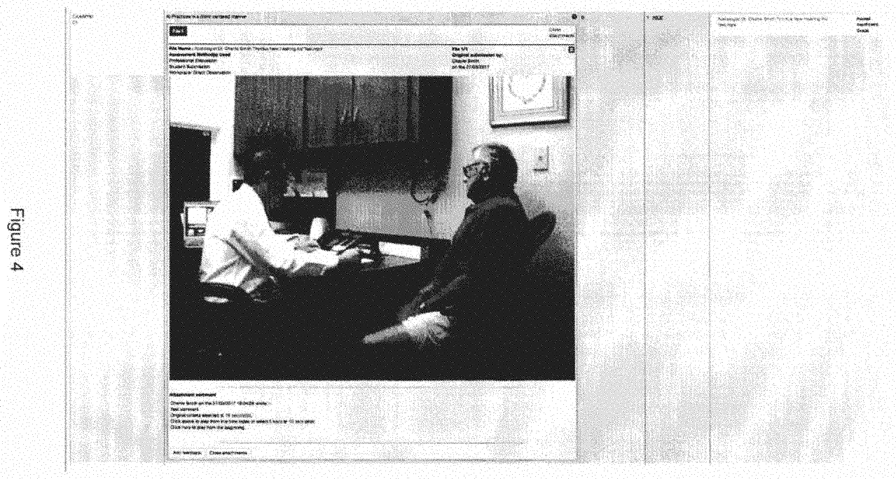

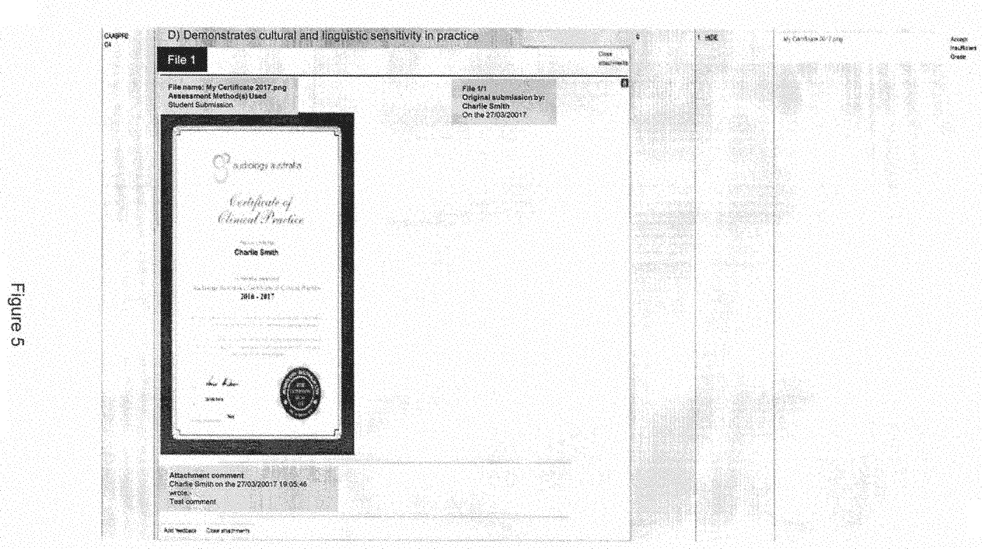

Another embodiment of an electronic profile 14 is shown in screen shots FIG. 4 and 5 which depicts documentary and video evidence 18 of achievement and activity to be assessed toward satisfying a candidate's competencies.

Regarding the evidence that may be submitted, in some embodiments, the system 1 is configured to allow for submission of evidence by only the candidate or representative of the candidate who is the subject of the profile, a third party or a combination of sources. For example, assuming system 1 is being utilized in a vocational education setting where each student has a profile and each student, along with various faculty and staff, have access to each student's profile. System 1 may be configured to allow the submission of evidence by the student and the various faculty and staff. Further, system 1 can be configured to allow the submitted evidence to be updated. For example, a student uploads video evidence showing a student completing a specific assignment into system 1 where it is determined that the student only has obtained partial competency in an ability. System 1 may allow the video evidence to be updated by the student or other party to demonstrate a complete competency in the ability.

Task and criteria module 20 may comprise a database 22 of various tasks and criteria requirements 24 for various competencies. For example, a welder desires to become recognized as a master welder. To obtain this recognition, the welder must have specific competencies in that field. These competencies can be further defined as a set of tasks and criteria, such as but not limited to, a consistent weld success rate documented by various testing methods, e.g. Magna Flux testing and X-ray testing; proficiency with welding techniques, e.g. Arc welding and knowledge of metal composition and configuration. Task and criteria module 20 may store these various tasks and criteria 24 and may be linked to the welder's profile. The welder will be able to determine what tasks and criteria are needed to be demonstrated before the recognition of master welder can be obtained.

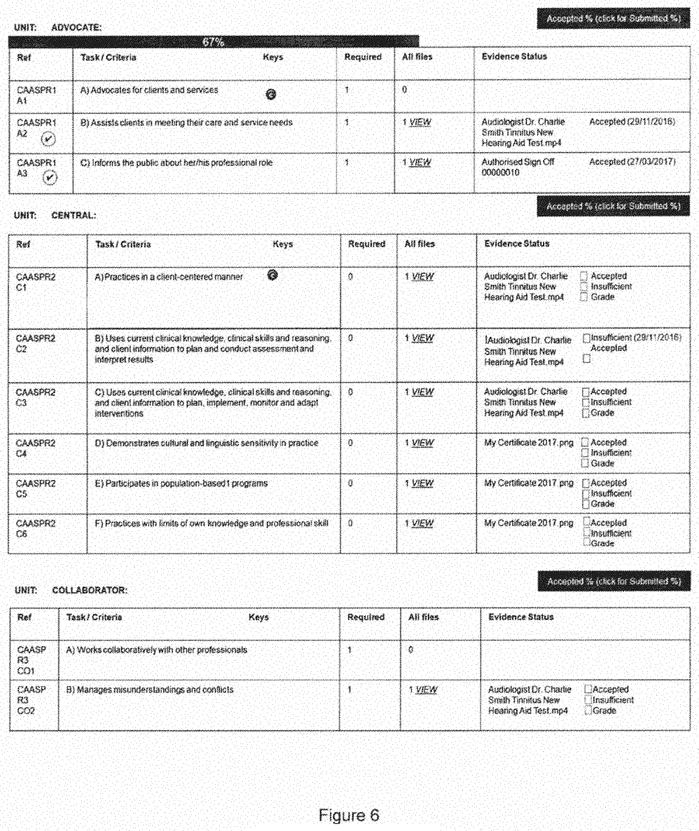

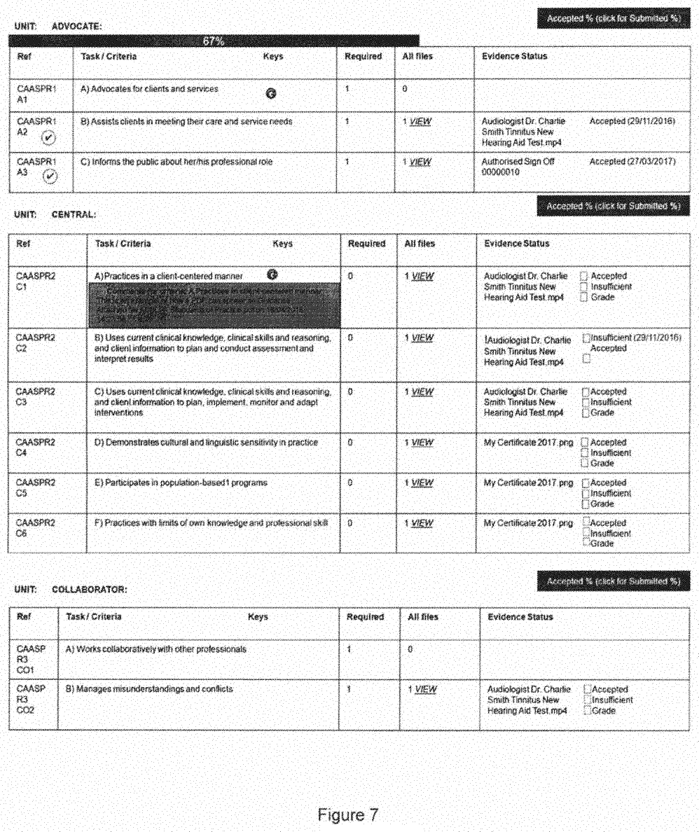

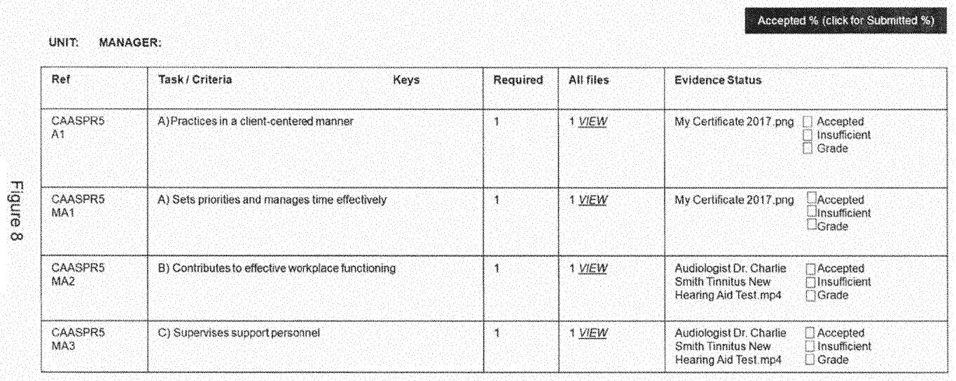

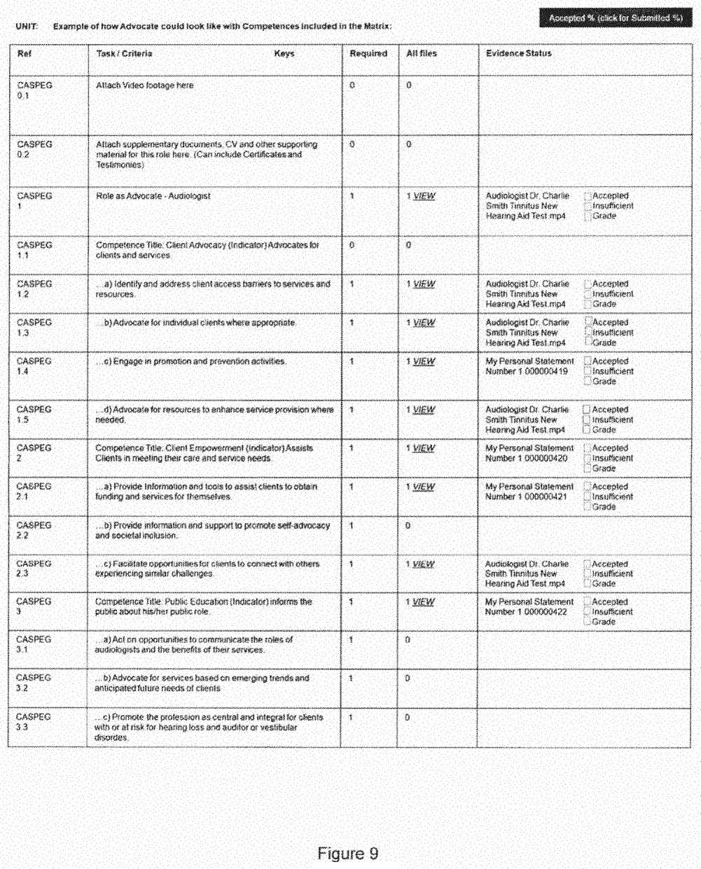

Embodiments of task and criteria module 20 are shown in screen shot FIGS. 6-9 which depict various tasks and criteria for various competencies.

Assessment module 30 allows an evaluator to review evidence 18 submitted on behalf of a candidate in order to assess the person's competency in an ability. Assessment module 30 provides the evaluator with the capability of reviewing evidence 18 in conjunction with the tasks and criteria 24 requirements for a particular competency. The evaluator can determine if the evidence is sufficient or not sufficient to establish that a candidate has satisfied the necessary tasks and/or criteria for a specific competency. The assessment module 30 further provides the evaluator the capability of providing a grade as related to a particular task or competency.

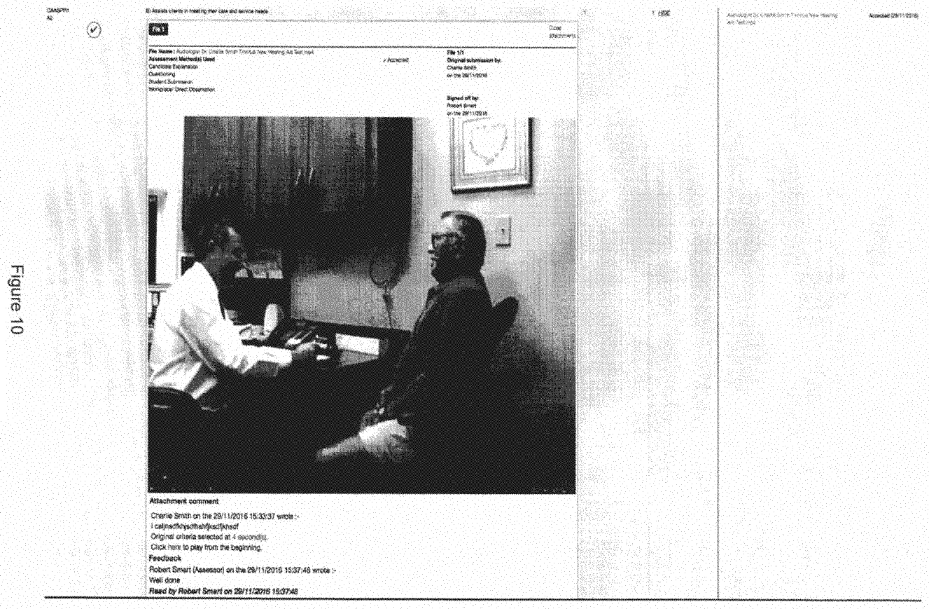

As an example embodiment of assessment module 30, as illustrated in screen shot FIG. 10, a video, that has been submitted as evidence 18 of competency of the task “Assists clients in meeting their care and service needs” has been determined to be accepted by the Assessor.

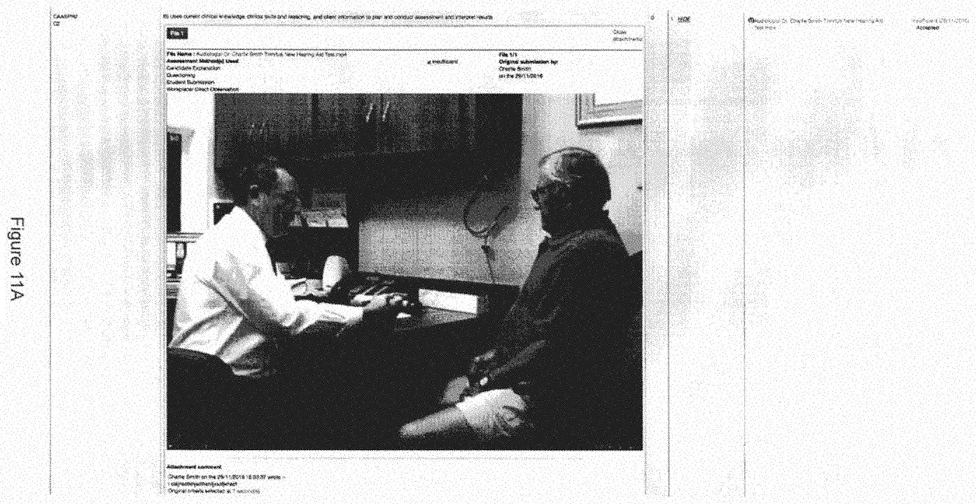

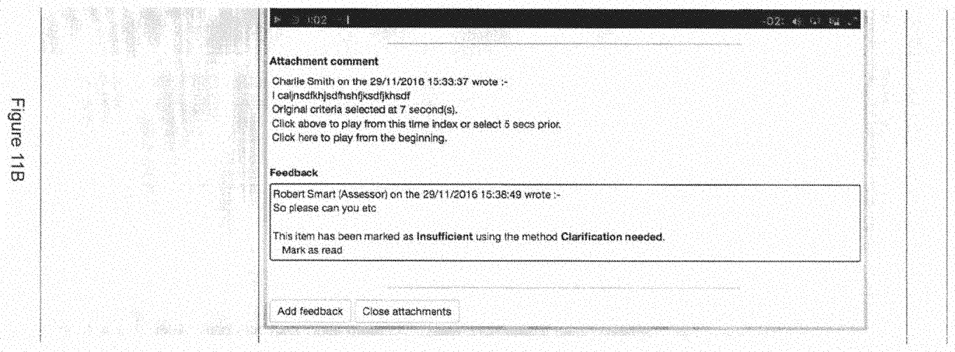

As an example embodiment of assessment module 30, as illustrated in screen shots FIGS. 11a and 11b, a video, that has been submitted as evidence 18 of competency of the task “Uses current clinical knowledge, clinical skills and reasoning, and client information to plan and conduct assessment and interpret results” has been determined to be insufficient by the Assessor.

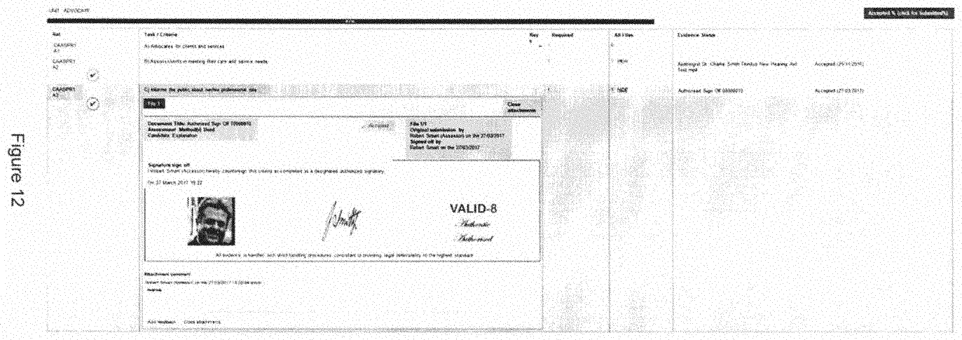

As embodiment of assessment module 30 provides the evaluator with the capability of acknowledging competency of a task and/or criteria without the submission of evidence. As illustrated in screen shot FIG. 12, an Assessor provides a signature sign-off for a particular criterion for a candidate.

An embodiment of assessment module 30 may include an evidence indexing component 32 for location indexing of documentary evidence and time indexing of video evidence. Evidence indexing component 32 provides the ability to identify a specific point in time in a video or a specific portion of a document to demonstrate the successful completion of the task catena requirement.

Evidence indexing component 32 may provide a link between the specific evidence and the specific task and/or criteria to allow for recall at a later time. This provides an efficient means by which to recall the specific evidence utilizes to establish the competency of a specific task and/or criteria.

Evidence indexing component 32 may allow a user to link an exact moment in a piece of video evidence or location within a document to provide instant recall of one or more competency indicators. The ability to link to these specific time references or documentary locations increases efficiency as such saves significant time to identify proof of skills, knowledge and ability.

For example, when a video is examined and reviewed, the Assessor, or another person reviewing the video, may select ail the relevant competency indicators at the time of observation in the video. The time frame for the competency indicators are identified and indexed. A link to the specific indexed time frames are created and embedded within the person's electronic profile. This allows for further review of the competency indicators without the need to review the entire video without having to pause, rewind or search the video. Further, this allows multiple links to he generated during one sitting or review, and as such increases efficiency.

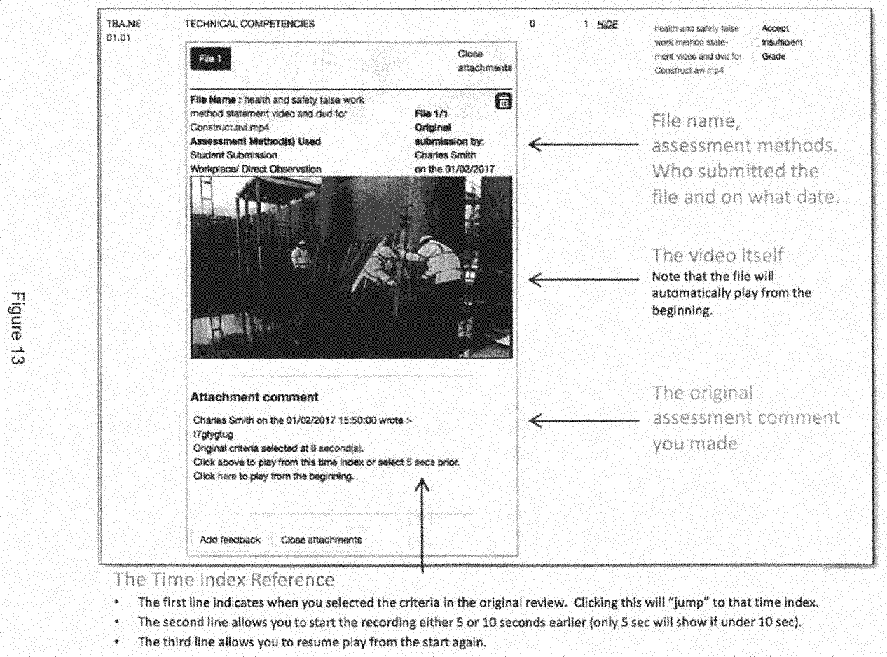

An embodiment of evidence indexing component 32, as illustrated in screen shot FIG. 13, demonstrates video evidence that has been submitted in connection with a particular task. Evidence indexing component 32 provides the Assessor with the capability to identify a particular time frame within the video that satisfies the particular task. In FIG. 13, the particular time frame is set at the “8 second” mark of the video.

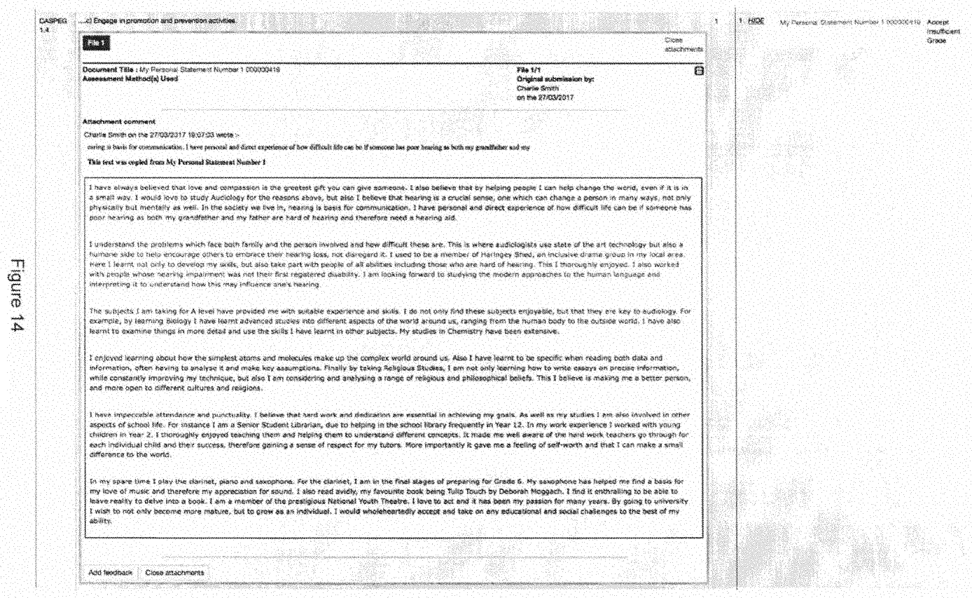

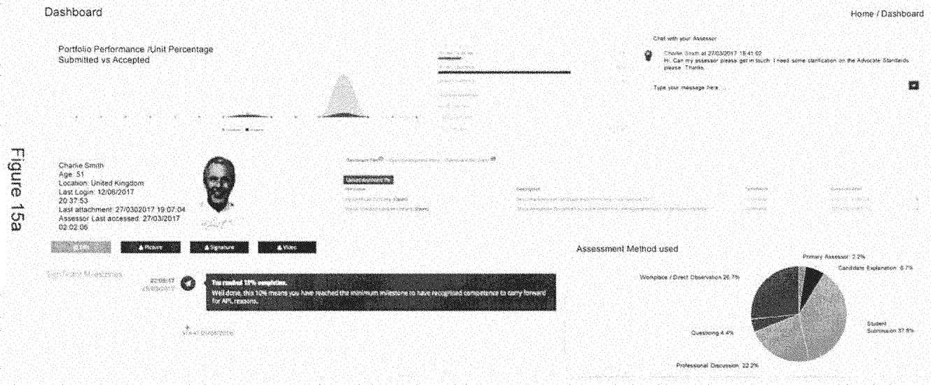

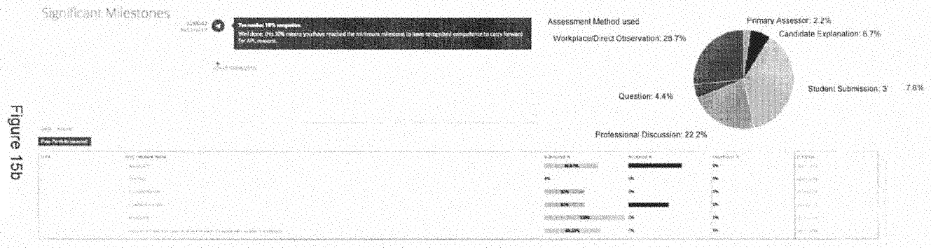

An embodiment of evidence indexing component 32, as illustrated in screen shot FIG. 14, demonstrates documentary evidence that has been submitted in connection with a particular task. Evidence indexing component 32 provides the Assessor with the capability to identify a particular section within the document that satisfies the particular task. In FIG. 13, the particular section is included within the highlighted portion of the document.

Since it is inefficient to continually review entire evidentiary submission at different times, evidence indexing component allows system 1 to be more efficient.

In another embodiment of the present invention, system 1 provides for a second layer of evaluation by a verifier who reviews the assessor's decisions and confirms those decisions or can challenge the decisions for further evaluation.

In another embodiment of the present invention, system 1 provides for an auto reference component This auto reference component automatically cross references multiple specific tasks and criteria. This cross referencing provides the evaluator with the ability to evaluate multiple tasks and criteria as a group. For example, an activity is common across multiple tasks and criteria. When an Assessor determines that the activity has been satisfied based on a specific piece of evidence, that activity within all the tasks and criteria with the common activity may be identified as being satisfied. Further, in conjunction with the evidence indexing component 32, the linked indexed evidence may be linked to each task and criteria at the same time. For example, once an Assessor indexes a specific time frame within a piece of video evidence, the indexed time frame may be linked to each task and criteria with the common activity. This embodiment allows system 1 to be more efficient.

In another embodiment of the present invention, system 1 provides for the automated development of task and criteria completion plans. System 1 may identify those tasks and criteria that not accepted and create a list of such tasks and criteria. As an example, assume system 1 is being utilized with a vocational school and is configured to ensure that the students have satisfied specific tasks and criteria tor the specific vocations in which are they are being trained. The development plan can be utilized to guide the student and the school into determining what skills that each student would need to complete to establish competency for the specific vocation.

In another embodiment of the present invention, system 1 provides for the evaluation of the evidence submitted through a natural language process or a text-to-speech process Keywords and other specific comments or notations may be utilized to help filter or screen persons. This may provide an efficient means to screen large number of applicants for a new job.

In another embodiment of the present invention, system 1 provides the capability for a holistic assessment by the assessor This capability allows the assessor to approve or not approve a group of tasks and criteria that are related, thus, allowing for a more efficient manner in which to record the competencies of a candidate.

In another embodiment of the present invention, system 1 provides for evidence to be provided through a mobile application. This evidence may be time stamped such that when they are uploaded to system 1, which can also be automatic or at a specific time, the videos cannot be altered. Thus, the video is a raw accurate representation.

Through the descriptions of the preceding embodiments, the present invention may be implemented by using hardware only or by using software and a necessary universal hardware platform. Based on such understandings, the technical solution of the present invention may be embodied in the form of a software product. The software product may be stored in a non-volatile or non-transitory storage medium, which can be a compact disk read-only memory (CD-ROM), USB flash disk, or a removable hard disk. The software product includes a number of instructions that enable a computer device (personal computer, server, or network device) to execute the methods provided in the embodiments of the present invention. For example, such an execution may correspond to a simulation of the logical operations as described herein. The software product may additionally or alternatively include number of instructions that enable a computer device to execute operations for configuring or programming a digital logic apparatus in accordance with embodiments of the present invention.

Although the present invention has been described with reference to specific features and embodiments thereof, it is evident that various modifications and combinations can be made thereto without departing from the invention. The specification and drawings are, accordingly, to be regarded simply as an illustration of the invention as defined by the appended claims; and are contemplated to cover any and all modifications, variations, combinations or equivalents that fall within the scope of the present invention.

1. A computer implemented method of conducting a competency-based assessment of a candidate, the method comprising:
a) providing a database comprising a plurality of electronic profiles, wherein each electronic profile is for a specific candidate and comprises a candidate identifier and a competency level;
b) updating an electronic profile of a specific candidate to include electronic evidence of achievement, wherein the evidence of achievement supports a specific competency;
c) indexing or annotating the electronic evidence based on a criterion to obtain processed electronic evidence;
d) defining a level of competency for the specific competency based on the processed evidence of achievement and
e) optionally, further comprising providing guidance to improve the level of competency and/or feedback,
wherein each electronic profile optionally comprises biographical information of a candidate, wherein the biographical information optionally comprises one or more of name, address, title, age and image of the candidate.

2. The computer implemented method of claim 1, wherein the candidate is an individual optionally selected from a human, non-human animal, a computer entity, or robotic candidate, an institute, an organization, a group or a company.

3. (canceled)

4. (canceled)

5. (canceled)

6. (cancelled)

7. The computer implemented method of claim 1, wherein the electronic evidence of an achievement comprises one or more of input text, electronic documents, images, videos, blog entries, multimedia presentation, audio recordings, health and fitness data and map coordinates and is optionally a result from a standardized test.

8. The computer implemented method of claim 1, wherein the specific competency is user defined, is selected from a pre-set list and/or is a requirement of an accreditation or program.

9. (canceled)

10. (canceled)

11. (canceled)

12. (canceled)

13. (canceled)

14. The computer implemented method of claim 1, wherein indexing or annotating the electronic evidence comprises reviewing the electronic evidence and bookmarking, highlighting a part of the evidence, time indexing and/or meta data tagging and wherein optionally details and optional links to bookmarks, highlights, time index, and/or meta data tagging is aggregated into a list in a dedicated section of the user interface.

15. (canceled)

16. The computer implemented method of claim 14, wherein the dedicated section is searchable and is optionally a bookmark pane, pop-up box, or separate dialog box.

17. (canceled)

18. The computer implemented method of claim 14, wherein bookmarks, highlights, time index, and/or meta data tagging are inserted using a mouse, touch pad or touch screen.

19. The computer implemented method of claim 14, wherein each bookmark in the bookmark pane is assigned one or more actions.

20. The computer implemented method of claim 19, wherein the evidence is an electronic document and the one or more actions comprises going to a specific page or passage in the document or wherein the evidence is a video or audio recording and the one or more actions comprises going to a specific frame in the video or point in the audio.

21. (canceled)

22. The computer implemented method of claim 19, wherein the one or more actions comprises opening a separate file and optionally wherein the separate file is opened in a new pane.

23. (canceled)

24. The computer implemented method of claim 22, wherein the separate file is an image file or text file.

25. (canceled)

26. (canceled)

27. (canceled)

28. (canceled)

29. The computer implemented method of claim 1, wherein the candidate is a candidate for employment or certification.

30. The computer implemented method of claim 1, wherein the candidate is a patient, optionally wherein the patient is undergoing treatment.

31. (canceled)

32. (canceled)

33. The computer implemented method of claim 1, wherein the method comprises repeating steps b) to d), and further comprising assessing for a change in the level of competency over time.

34. (canceled)

35. (canceled)

36. (canceled)

37. The computer implemented method of claim 33, wherein the change is an improvement in the level of competency.

38. The computer implemented method of claim 37, wherein the improvement is a measure of effectiveness a treatment or training regime.

39. The computer implemented method of claim 33, wherein the change is a decrease in the level of competency.

40. The computer implemented method of claim 39, wherein the decrease is a measure of disease progression and/or age-related changes.

41. (canceled)

42. (canceled)

43. (canceled)

44. (canceled)

45. (canceled)

46. The computer implemented method of claim 1, wherein the updating of the electronic profile of the specific candidate to include electronic evidence of achievement and/or annotating or indexing the evidence is completed by third party, optionally wherein the third party is known to the candidate or is an independent assessor.

47. (canceled)

48. (canceled)

49. (canceled)

50. (canceled)

51. (canceled)

52. (canceled)

53. (canceled)

54. (canceled)

55. (canceled)

56. (canceled)
